(19) United States
(12) Patent Application Publication
(10) Pub. No.: US 2009/0054747 A1
(43) Pub. Date: Feb. 26, 2009

(54) Method and system for providing analyte sensor tester isolation

(75) Inventor: Martin J. Fennell, Concord, CA (US)

Correspondence Address: JACKSON & CO., LLP, 6114 LA SALLE AVENUE, #507, OAKLAND, CA, 94611-2802, US

(73) Assignee: Abbott Diabetes Care, Inc., Alameda, CA (US)

(21) Appl. No.: 11/264,118

(22) Filed: Oct. 31, 2005

(51) Int. Cl. A61B 5/1477

(52) U.S. Cl. 600/316

(57) ABSTRACT

Method and apparatus for providing electrical isolation between devices in batch testing process during manufacturing procedure is provided.

### BACKGROUND

Analyte, e.g., glucose, monitoring systems including continuous and discrete monitoring systems generally include a small, lightweight battery powered and microprocessor controlled system which is configured to detect signals proportional to the corresponding measured analyte levels using an electrometer, and RF signals to transmit the collected data. One aspect of certain glucose monitoring systems include a transcutaneous or subcutaneous analyte sensor configuration which is, for example, partially mounted on the skin of a subject whose glucose level is to be monitored. The sensor cell may use a two or three-electrode (work, reference and counter electrodes) configuration driven by a controlled potential (potentiostat) analog circuit connected through a contact system.

The analyte sensor may be configured so that a portion thereof is placed under the skin of the patient so as to detect the analyte levels of the patient, and another portion of segment of the analyte sensor that is in communication with the transmitter unit. The transmitter unit is configured to transmit the analyte levels detected by the sensor over a wireless communication link such as an RF (radio frequency) communication link. To transmit signals, the transmitter unit requires a power supply such as a battery. Generally, batteries have a limited life span and require periodic replacement. Depending on the power consumption of the transmitter unit, the power supply in the transmitter unit may require frequent replacement, or the transmitter unit may require replacement (e.g, disposable power supply such as disposable battery).

For effective management and monitoring analytes, patients generally are required to replace the analyte sensor frequently at a predetermined intervals such as after 3 days, 5 days or 7 days of continuous use, for example. On the other hand, the transmitter unit and the electronics therein are configured for extended use, and as such, provide durability and structural integrity against daily usage and normal wear and tear.

Given the disposable component of the continuous monitoring systems, a typical patient will generally use a large number of sensors, and indeed, within the overall continuous monitoring system, the analyte sensors are one of the components that require frequent replacement.

As such, from manufacturing perspective, it is important to have a system for manufacturing and testing the sensors for functionality so as to achieve scalability and cost effectiveness. Indeed, given the volume of the analyte sensors even a single patient using the continuous monitoring system will likely require, it is important to minimize the potential sensor failures during the manufacturing process, as well as to implement a system for manufacture that provides for parallel or substantially concurrent testing of the sensors (rather than sequentially one at a time), which would significantly add to the manufacturing scalability and cost effectiveness.

For example, analyte sensors for use in the continuous monitoring system may be tested during the manufacturing process in the same test fluid (e.g., glucose concentration fluid). The test fluid is generally electrically conductive, and thus, to avoid adjacent sensor being potentially adversely affected, each sensor must be electrically isolated from each other during the testing process and while substantially immersed in the test fluid.

In view of the foregoing, it would be desirable to have a system of manufacturing and/or testing analyte sensors or any other types of mass producible components of an overall system, that provides manufacturing scalability and cost effective production.

### SUMMARY OF THE INVENTION

In view of the foregoing, in accordance with the various embodiments of the present invention, there is provided a method and system for minimizing potential sensor failures during the manufacturing and/or testing process of the analyte sensors. More specifically, there is provided a method and system in accordance with the various embodiments of the present invention to electrically isolate the analyte sensors which are electrically conductive with respect to each other during the manufacturing and testing process.

In this manner, in accordance with the various embodiments of the present invention, the testing of each analyte sensor during the manufacturing process may be performed and, the process of such testing to not substantially impact the integrity of other analyte sensors in a concurrent manufacturing process by electrically isolating each analyte sensor with respect to each of the other analyte sensors in the manufacturing process and which are in electrically conductive medium.

Indeed, within the scope of the present invention, it is possible to achieve the pico-amp level or better of sensor isolation during the testing procedure, where the sensor may be at a range below 50 nA.

### BRIEF DESCRIPTION OF THE DRAWINGS

FIG. 1 illustrates a block diagram of a data monitoring and management system for practicing one embodiment of the present invention;

FIG. 2 is a block diagram of the transmitter unit of the data monitoring and management system shown in FIG. 1 in accordance with one embodiment of the present invention;

FIG. 3 illustrates a sensor tester isolation system in accordance with one embodiment of the present invention;

FIG. 4 illustrates a detailed view of the sensor tester isolation system of FIG. 3 in accordance with one embodiment of the present invention;

FIG. 5 illustrates a sensor tester isolation system with including testing unit power supplies in accordance with another embodiment of the present invention;

FIG. 6 illustrates a sensor tester isolation system including a transformer in accordance with yet another embodiment of the present invention;

FIG. 7 is a flow chart illustrating the sensor tester isolation process in accordance with one embodiment of the present invention; and

FIG. 8 is a flow chart illustrating the sensor tester isolation process in accordance with another embodiment of the present invention.

### DETAILED DESCRIPTION

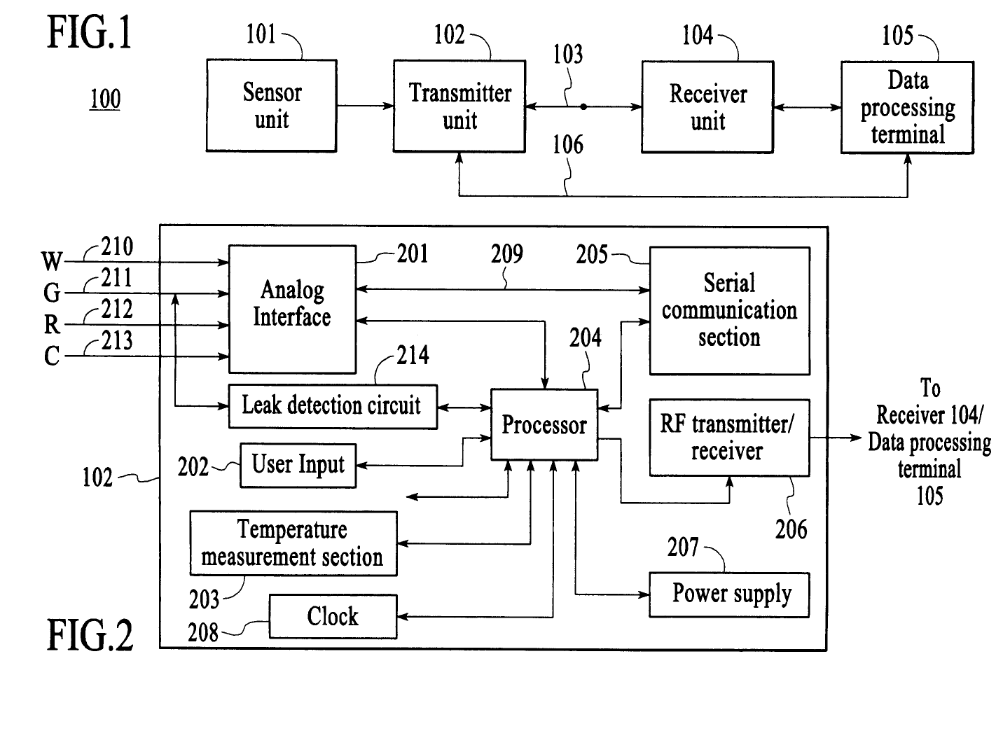

FIG. 1 illustrates a data monitoring and management system such as, for example, an analyte (e.g., glucose) monitoring system 100 in accordance with one embodiment of the present invention. The subject invention is further described primarily with respect to a glucose monitoring system for convenience and such description is in no way intended to limit the scope of the invention. It is to be understood that the analyte monitoring system may be configured to monitor a variety of analytes, e.g., lactate, and the like.

Indeed, analytes that may be monitored include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored.

The glucose monitoring system 100 includes a sensor 101, a transmitter 102 coupled to the sensor 101, and a receiver 104 which is configured to communicate with the transmitter 102 via a communication link 103. The receiver 104 may be further configured to transmit data to a data processing terminal 105 for evaluating the data received by the receiver 104. Moreover, the data processing terminal in one embodiment may be configured to receive data directly from the transmitter 102 via a communication link 106 which may optionally be configured for bi-directional communication.

Only one sensor 101, transmitter 102, communication link 103, receiver 104, and data processing terminal 105 are shown in the embodiment of the glucose monitoring system 100 illustrated in FIG. 1. However, it will be appreciated by one of ordinary skill in the art that the glucose monitoring system 100 may include one or more sensor 101, transmitter 102, communication link 103, receiver 104, and data processing terminal 105, where each receiver 104 is uniquely synchronized with a respective transmitter 102. Moreover, within the scope of the present invention, the glucose monitoring system 100 may be a continuous monitoring system, or semi-continuous, or a discrete monitoring system.

In one embodiment of the present invention, the sensor 101 is physically positioned in or on the body of a user whose glucose level is being monitored. The sensor 101 may be configured to continuously sample the glucose level of the user and convert the sampled glucose level into a corresponding data signal for transmission by the transmitter 102. In one embodiment, the transmitter 102 is mounted on the sensor 101 so that both devices are positioned on the user's body. The transmitter 102 performs data processing such as filtering and encoding on data signals, each of which corresponds to a sampled glucose level of the user, for transmission to the receiver 104 via the communication link 103.

In one embodiment, the glucose monitoring system 100 is configured as a one-way RF communication path from the transmitter 102 to the receiver 104. In such embodiment, the transmitter 102 transmits the sampled data signals received from the sensor 101 without acknowledgement from the receiver 104 that the transmitted sampled data signals have been received. For example, the transmitter 102 may be configured to transmit the encoded sampled data signals at a fixed rate (e.g., at about one minute intervals) after the completion of the initial power on procedure. Likewise, the receiver 104 may be configured to detect such transmitted encoded sampled data signals at predetermined time intervals. Alternatively, the glucose monitoring system 100 may be configured with a bi-directional RF (or otherwise) communication between the transmitter 102 and the receiver 104.

Additionally, in one aspect, the receiver 104 may include two sections. The first section is an analog interface section that is configured to communicate with the transmitter 102 via the communication link 103. In one embodiment, the analog interface section may include an RF receiver and an antenna for receiving and amplifying the data signals from the transmitter 102, which are thereafter, demodulated with a local oscillator and filtered through a band-pass filter. The second section of the receiver 104 is a data processing section which is configured to process the data signals received from the transmitter 102 such as by performing data decoding, error detection and correction, data clock generation, and data bit recovery.

In operation, upon completing the power-on procedure, the receiver 104 is configured to detect the presence of the transmitter 102 within its range based on, for example, the strength of the detected data signals received from the transmitter 102 or a predetermined transmitter identification information. Upon successful synchronization with the corresponding transmitter 102, the receiver 104 is configured to begin receiving from the transmitter 102 data signals corresponding to the user's detected glucose level. More specifically, the receiver 104 in one embodiment is configured to perform synchronized time hopping with the corresponding synchronized transmitter 102 via the communication link 103 to obtain the user's detected glucose level.

Referring again to FIG. 1, the data processing terminal 105 may include a personal computer, a portable computer such as a laptop or a handheld device (e.g., personal digital assistants (PDAs)), and the like, each of which may be configured for data communication with the receiver via a wired or a wireless connection. Additionally, the data processing terminal 105 may further be connected to a data network (not shown) for storing, retrieving and updating data corresponding to the detected glucose level of the user.

Within the scope of the present invention, the data processing terminal 105 may include an infusion device such as an insulin infusion pump or the like, which may be configured to administer insulin to patients, and which may be configured to communicate with the receiver unit 104 for receiving, among others, the measured glucose level. Alternatively, the receiver unit 104 may be configured to integrate an infusion device therein so that the receiver unit 104 is configured to administer insulin therapy to patients, for example, for administering and modifying basal profiles, as well as for determining appropriate boluses for administration based on, among others, the detected glucose levels received from the transmitter 102.

Additionally, the transmitter 102, the receiver 104 and the data processing terminal 105 may each be configured for bi-directional wireless communication such that each of the transmitter 102, the receiver 104 and the data processing terminal 105 may be configured to communicate (that is, transmit data to and receive data from) with each other via the wireless communication link 103. More specifically, the data processing terminal 105 may in one embodiment be configured to receive data directly from the transmitter 102 via the communication link 106, where the communication link 106, as described above, may be configured for bi-directional communication. In this embodiment, the data processing terminal 105 which may include an insulin pump, may be configured to receive the glucose signals from the transmitter 102, and thus, incorporate the functions of the receiver 103 including data processing for managing the patient's insulin therapy and glucose monitoring. In one embodiment, the communication link 103 may include one or more of an RF communication protocol, an infrared communication protocol, a Bluetooth enabled communication protocol, an 802.11x wireless communication protocol, or an equivalent wireless communication protocol which would allow secure, wireless communication of several units (for example, per HIPPA requirements) while avoiding potential data collision and interference.

FIG. 2 is a block diagram of the transmitter of the data monitoring and detection system shown in FIG. 1 in accordance with one embodiment of the present invention. Referring to the Figure, the transmitter 102 in one embodiment includes an analog interface 201 configured to communicate with the sensor 101 (FIG. 1), a user input 202, and an optional temperature detection section 203, each of which is operatively coupled to a transmitter processor 204 such as a central processing unit (CPU). As can be seen from FIG. 2, there are provided four contacts, three of which are electrodes—work electrode (W) 210, guard contact (G) 211, reference electrode (R) 212, and counter electrode (C) 213, each operatively coupled to the analog interface 201 of the transmitter 102 for connection to the sensor unit 201 (FIG. 1). In one embodiment, each of the work electrode (W) 210, guard contact (G) 211, reference electrode (R) 212, and counter electrode (C) 213 may be made using a conductive material that is either printed or etched, for example, such as carbon which may be printed, or metal foil (e.g., gold) which may be etched.

Further shown in FIG. 2 are a transmitter serial communication section 205 and an RF transmitter 206, each of which is also operatively coupled to the transmitter processor 204. Moreover, a power supply 207 such as a battery is also provided in the transmitter 102 to provide the necessary power for the transmitter 102. Additionally, as can be seen from the Figure, clock 208 is provided to, among others, supply real time information to the transmitter processor 204.

In one embodiment, a unidirectional input path is established from the sensor 101 (FIG. 1) and/or manufacturing and testing equipment to the analog interface 201 of the transmitter 102, while a unidirectional output is established from the output of the RF transmitter 206 of the transmitter 102 for transmission to the receiver 104. In this manner, a data path is shown in FIG. 2 between the aforementioned unidirectional input and output via a dedicated link 209 from the analog interface 201 to serial communication section 205, thereafter to the processor 204, and then to the RF transmitter 206. As such, in one embodiment, via the data path described above, the transmitter 102 is configured to transmit to the receiver 104 (FIG. 1), via the communication link 103 (FIG. 1), processed and encoded data signals received from the sensor 101 (FIG. 1). Additionally, the unidirectional communication data path between the analog interface 201 and the RF transmitter 206 discussed above allows for the configuration of the transmitter 102 for operation upon completion of the manufacturing process as well as for direct communication for diagnostic and testing purposes.

As discussed above, the transmitter processor 204 is configured to transmit control signals to the various sections of the transmitter 102 during the operation of the transmitter 102. In one embodiment, the transmitter processor 204 also includes a memory (not shown) for storing data such as the identification information for the transmitter 102, as well as the data signals received from the sensor 101. The stored information may be retrieved and processed for transmission to the receiver 104 under the control of the transmitter processor 204. Furthermore, the power supply 207 may include a commercially available battery.

The transmitter 102 is also configured such that the power supply section 207 is capable of providing power to the transmitter for a minimum of about three months of continuous operation after having been stored for about eighteen months in a low-power (non-operating) mode, where such time periods are for exemplary purposes only and are in no way intended to limit the scope of the invention. In one embodiment, this may be achieved by the transmitter processor 204 operating in low power modes in the non-operating state, for example, drawing no more than approximately 1 μA of current. Indeed, in one embodiment, the final step during the manufacturing process of the transmitter 102 may place the transmitter 102 in the lower power, non-operating state (i.e., post-manufacture sleep mode). In this manner, the shelf life of the transmitter 102 may be significantly improved. Moreover, as shown in FIG. 2, while the power supply unit 207 is shown as coupled to the processor 204, and as such, the processor 204 is configured to provide control of the power supply unit 207, it should be noted that within the scope of the present invention, the power supply unit 207 is configured to provide the necessary power to each of the components of the transmitter unit 102 shown in FIG. 2.

Referring back to FIG. 2, the power supply section 207 of the transmitter 102 in one embodiment may include a rechargeable battery unit that may be recharged by a separate power supply recharging unit so that the transmitter 102 may be powered for a longer period of usage time. Moreover, in one embodiment, the transmitter 102 may be configured without a battery in the power supply section 207, in which case the transmitter 102 may be configured to receive power from an external power supply source (for example, a battery) as discussed in further detail below.

Referring yet again to FIG. 2, the optional temperature detection section 203 of the transmitter 102 is configured to monitor the temperature of the skin near the sensor insertion site. The temperature reading may be used to adjust the glucose readings obtained from the analog interface 201. The RF transmitter 206 of the transmitter 102 may be configured for operation in the frequency band of 315 MHz to 322 MHz, for example, in the United States. Further, in one embodiment, the RF transmitter 206 may be configured to modulate the carrier frequency by performing Frequency Shift Keying and Manchester encoding. In one embodiment, the data transmission rate is about 19,200 symbols per second, with a minimum transmission range for communication with the receiver 104.

Referring yet again to FIG. 2, also shown is a leak detection circuit 214 coupled to the guard electrode (G) 211 and the processor 204 in the transmitter 102 of the data monitoring and management system 100. The leak detection circuit 214 in accordance with one embodiment of the present invention may be configured to detect leakage current in the sensor 101 to determine whether the measured sensor data are corrupt or whether the measured data from the sensor 101 is accurate.

Additional detailed description of the continuous glucose monitoring system, its various components including the functional descriptions of the transmitter are provided in U.S. Pat. No. 6,175,752 issued Jan. 16, 2001 entitled “Analyte Monitoring Device and Methods of Use”, and in application Ser. No. 10/745,878 filed Dec. 26, 2003 entitled “Continuous Glucose Monitoring System and Methods of Use”, each assigned to the Assignee of the present application.

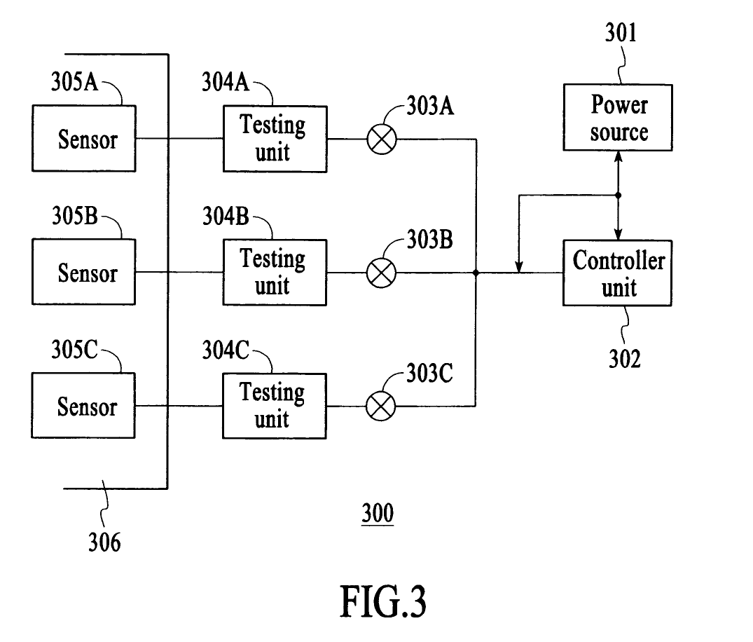

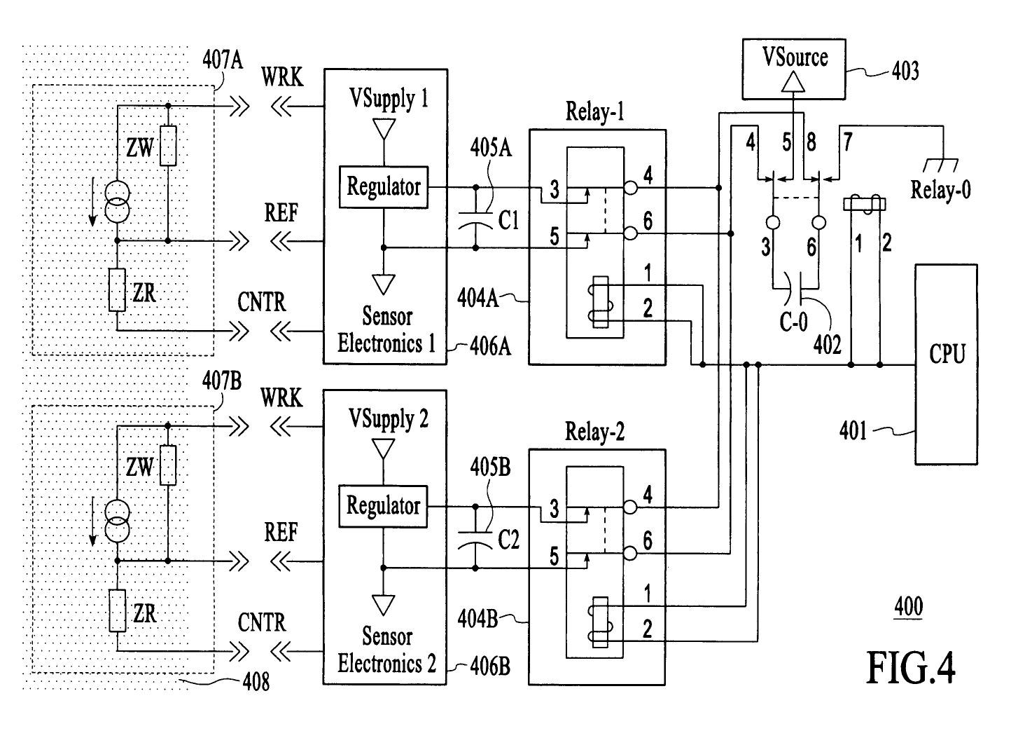

FIG. 3 illustrates a sensor tester isolation system in accordance with one embodiment of the present invention. Referring to FIG. 3, the tester isolation system 300 in one embodiment of the present invention includes a controller unit 302 operatively coupled to a power source 301. The controller unit 302 may be configured to operatively control the power source 301 providing power to a plurality of relay circuits 303A, 303B, 303C. In one embodiment, the relay circuits 303A, 303B, 303C may each include a relay switch each of which, under the control of the controller unit 302, is configured to turn on or off to couple the power source 301 to a respective one of a plurality of testing units 304A, 304B, 304C.

Indeed, as can be seen from FIG. 3, the tester isolation system 300 in one embodiment includes a plurality of sensors 305A, 305B, 305C, each of which are substantially immersed in an electrically conductive test fluid 306 during the manufacturing and testing process of the analyte sensors 305A, 305B, 305C. Moreover, each of the analyte sensors 305A, 305B, 305C are each respectively coupled to a corresponding one of the testing units 304A, 304B, 304C, each of which are configured to perform testing procedures on the respective analyte sensor 305A, 305B, 305C.

More specifically, in one embodiment, the testing units 304A, 304B, 304C are each configured to test for linearity of the sensor electrodes as a function of the glucose concentration, the sensor measurement scale determination, and sensor stability. Indeed, the testing units 304A, 304B, 304C may be configured to test the respective analyte sensors 305A, 305B, 305C for linearity of the sensor electrodes (working, reference, and counter electrodes, for example) as a function of glucose concentration, which may be performed by varying the concentration of the glucose in the test fluid 306 by a predetermined level, and observing the response from the sensor electrodes.

In performing the sensor measurement scale determination, each of the testing units 304A, 304B, 304C may be configured to determine the full measurement scale of the respective sensor 305A, 305B, 305C by increasing the concentration of the glucose level in the test fluid 306 to the highest level of intended measurement, and then determining whether the respective sensors 305A, 305B, 305C are detecting the highest glucose level of the test fluid. Also, in testing for stability of the sensors 305A, 305B, 305C, the glucose concentration of the test fluid 306 may be maintained at a predetermined level for a given period of time (for example, 1 day, 3 days, or 7 days), and measuring the detected signals at the sensor electrodes to determine whether the predetermined glucose level is detected by the analyte sensor electrodes.

Referring back to FIG. 3, for each of the testing units 304A, 304B, 304C to properly perform the testing procedures for the respectively coupled analyte sensor 305A, 305B, 305C, and retain electrical isolation of the analyte sensors 305A, 305B, 305C from each other while immersed in the electrically conductive test fluid 306, the controller 302 is configured to sequentially operate the relay circuits 303A, 303B, 303C so as to provide the necessary power to each of the testing units 304A, 304B, 304C sequentially from the power source 301.

In this manner, in one embodiment of the present invention, the tester isolation system 300 may use a single power supply such as the power source 301 to provide power to each of the testing units 304A, 304B, 304C, while also providing electrical isolation of the analyte sensors 305A, 305B, 305C during the testing processes. Indeed, even though the analyte sensors 305A, 305B, 305C are immersed in the same electrically conductive test fluid 306, it is possible to provide isolation of each analyte sensor 305A, 305B, 305C with respect to each other during the testing process using a single power supply. As such, the manufacturing and testing process of the sensors may be streamlines and achieved with improved time and cost efficiency.

Referring again to FIG. 3, while there are shown three analyte sensors 305A, 305B, 305C, each coupled to a respective one of the three testing units 304A, 304B, 304C, and which in turn are respectively coupled to one of the three relay circuits 303A, 303B, 303C, within the scope of the present invention, additional sensors can be tested during the same testing process, and likewise provided in the same testing fluid 306, each of which may be provided with a respective testing unit and a relay circuit, where the relay circuit is operatively coupled to the controller unit 302 and the power source 301. Indeed, within the scope of the present invention, it is possible to test for hundreds of analyte sensors or more in the same electrically conductive test fluid using a single power source, and while achieving electrical isolation of each of the analyte sensors with respect to each other.

In this manner, the tester isolation system 100 may be scaled up for mass production and testing of analyte sensors or other devices to the extent that the testing units for performing the sensor tests have sufficient charge or power to perform the testing procedures. For example, in one embodiment, the tester isolation system 100 may be divided into about 16 testing units or channels per testing cycle based on the power provided by the power source 301. Moreover, while analyte sensors are described in the testing process in conjunction with FIG. 3, within the scope of the present invention, any other types of devices may be tested in similar manner to obtain electrical isolation during testing and manufacturing processes.

FIG. 4 illustrates a detailed view of the sensor tester isolation system of FIG. 3 in accordance with one embodiment of the present invention. Referring to FIG. 4, tester isolation system 400 in one embodiment of the present invention includes a control unit 401 such as a central processing unit (CPU). Also provided is a power supply V 403 (shown as Vsource) which is coupled to capacitor 402 (shown as Cfly), and under the control of the control unit 401, the capacitor 402 is configured to be charged to the voltage level of the power supply 403 at the beginning of the testing process.

In one embodiment, the size of the capacitor 402 may vary depending upon the desired time frame for performing the sequential testing. For example, the smaller the size of the capacitor 402, the faster the testing of the sensors need to be performed so that all of the test capacitors 405A, 405B may be sequentially charged to the appropriate level during the testing process, and while the capacitor 402 still has sufficient charge.

Referring to FIG. 4, also shown in the Figure are relay circuits 404A, 404B, each of which are coupled to a first test capacitor 405A and a second test capacitor 405B. In turn, each of the first test capacitor 405A and the second test capacitor 405B are respectively coupled to a first sensor test unit 406A, and a second sensor test unit 406B. Moreover, as can be further seen from FIG. 4, each of the first sensor test unit 406A the second sensor test unit 406B are in turn coupled to a first sensor 407A and a second sensor 407B, each of which are provided in an electrically conductive test fluid 408. In one embodiment, each of the first and second test capacitor 405A, 405B include supercaps at approximately 0.68 Farads.

As shown in FIG. 4, the control unit 401 is configured to initially charge the capacitor 402 substantially to the voltage level of the power source 403 (Vsource, for example, at 5 Volts). After charging the capacitor 402, the control unit 401 is configured to sequentially charge the first test capacitor 405A and the second test capacitor 405B by energizing the respective relay circuits 404A, 404B. The first and the second sensor test units 406A, 406B are each configured in one embodiment with a voltage regulator which is configured to receive the power from the respective first and second test capacitors 405A, 405B, so as to regulate the voltage within the respective sensor test unit 406A, 406B to a suitable level for performing the testing procedures.

In this manner, the tester isolation system 400 in one embodiment of the present invention may be configured to prevent current from flowing between the sensors 407A, 407B during the testing process by the respective sensor test units 406A, 406B, while immersed in the electrically conductive test fluid 408. Moreover, the electrical isolation between the sensors 407A, 407B may be achieved using one power source (e.g., the capacitor 402) under the operational control of the control unit 401.

Referring still again to FIG. 4, while only two analyte sensors 407A, 407B are shown, within the scope of the present invention, multiple sensors may be tested at the same time while substantially immersed in the same test fluid 408, where each additional sensor is coupled to a sensor test unit that is coupled to a respective test capacitor and a relay circuit, where the relay circuit is substantially controlled by the control unit 401.

In the manner described above, in one embodiment of the present invention, under the control of the control unit 401, the capacitor 402 is initially charged to the voltage level of the power source 403 (Vsource), and after disconnecting the connection between the power source 403 and the capacitor 402, the control unit 401 sequentially turns on the relay circuit 404A and 404B such that the stored charge in the capacitor 402 is provided sequentially to the first and second test capacitors 405A, 405B. Accordingly, electrical isolation between the analyte sensors 407A and 407B immersed in the electrically conductive test fluid 408 is achieved.

In one embodiment, the capacitor 402 may be about 1.0 Farad, and each of the test capacitors 405A, 405B for each test channel may be about 0.47 Farad for a load of about 2.5 mA per test channel. Within the scope of the present invention, the size of these capacitors may be adjusted if, for example, they were charged at a higher frequency. Also, in one embodiment, the control unit 401 may be configured to sequentially energize the relay circuits 404A and 404B such that the capacitor 402 is coupled to each test channel for approximately 250 milliseconds, and then charged up for 250 milliseconds coupled to the power source 301. In this manner for example, in one embodiment, the control unit 401 charges up the capacitor 402 for 250 ms, and then couples to the first relay circuit 404A for 250 ms. Then, the capacitor 402 is charged for another 250 ms by coupling it to the power source 301, and then is coupled to the second relay circuit 404B for another 250 ms. This process continues until each of the relay circuits 404A, 404B, 404C have bee coupled to the capacitor 402.

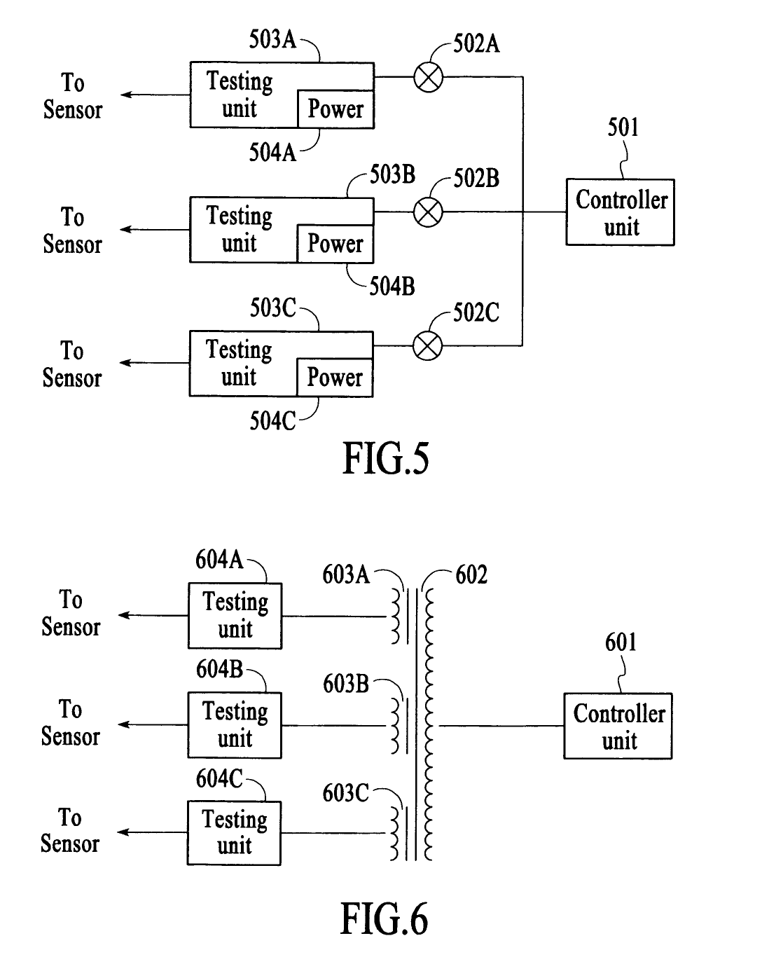

FIG. 5 illustrates a sensor tester isolation system including testing unit power supplies in accordance with another embodiment of the present invention. Referring to the FIG. 5, in one embodiment of the present invention, there is provided a controller unit 501 which is operatively coupled to a plurality of switch units 502A, 502B, 502C, each of which are coupled to a respective one of a plurality of testing units 503A, 503B, 503C. Moreover, as can be seen from FIG. 5, each testing unit 503A, 503B, 503C are provides with a respective power supply 504A, 504B, 504C.

In this manner, the controller unit 501 is configured to control the sequential switching of the plurality of the switch units 502A, 502B, 502C, and upon the switch activation operation by the controller unit 501, the respective one of the testing units 503A, 503B, 503C is configured to perform the testing procedure on the sensor that is coupled to the respective one of the testing units 503A, 503B 503C, powered by the corresponding power supply 504A, 504B, 504C of the respective testing unit 403A, 503B, 503C. In this manner, under the control of the controller unit 501, electrical isolation between the sensors during the testing procedure may be achieved.

FIG. 6 illustrates a sensor tester isolation system including a transformer in accordance with yet another embodiment of the present invention. Referring to FIG. 6, and contrasted with the embodiment shown in FIG. 5, rather than providing a separate power supply 504A, 504B, 504C for each of the testing units 503A, 503B, 503C, in the embodiment shown in FIG. 6, there is provided a transformer operatively coupled between controller unit 601 and each of the testing units 604A, 604B, 604C, where each of the testing units 604A, 604B, 604C are operatively coupled with a respective isolated secondary windings 603A, 603B, 603C for the transformer to cooperate with the primary winding 602 of the transformer.

In this manner, power may be provided to the testing units 604A, 604B, 604C under the control of the controller unit 601 so as to obtain electrical isolation between each sensor coupled to a respective one of the testing units 604A, 604B, 604C during the sensor testing procedures in manufacturing.

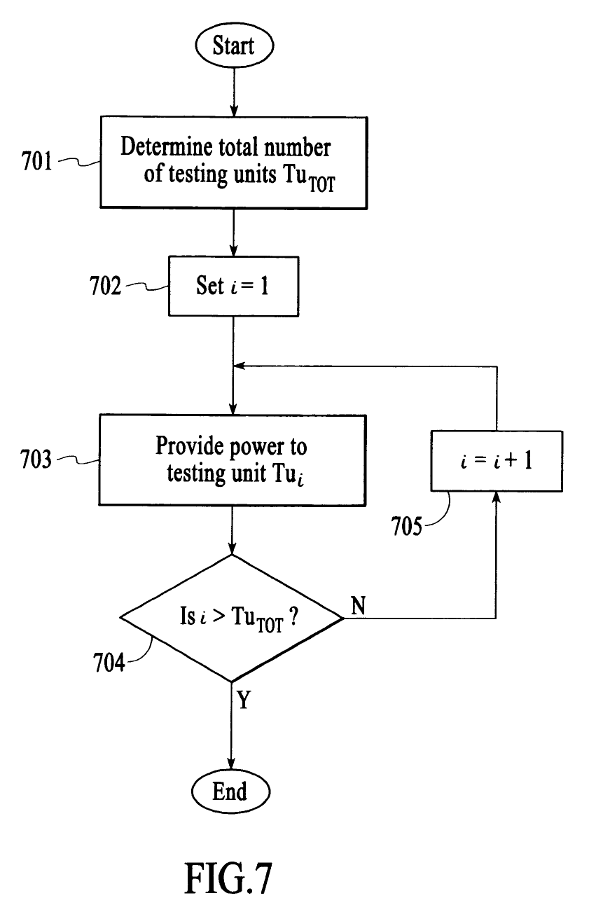

FIG. 7 is a flow chart illustrating the sensor tester isolation process in accordance with one embodiment of the present invention. Referring to FIG. 7, at step 701, the total number of the testing units TUtot is determined so as to determine the number of the analyte sensors in the testing cycle during the batch manufacturing and processing. Thereafter, at step 702, a variable “i” is initialized to one (“1”), and at step 703, power is provided to testing unit TUi, where variable i is set at 1 at step 702. In one embodiment, and referring to FIG. 3, step 703 may be performed by or under the control of the controller unit 302 to provide power from the power source 301 to testing unit 304A.

Referring back to FIG. 7, after providing power to testing unit TUi at step 703, it is determined whether the variable i is equal to or greater than the total number of testing units TUtot determined at step 701. If the variable i is equal to or greater than the total number of testing units TUtot determined at step 701, then it is determined that the testing procedure in the last sensor in the manufacturing batch of sensors is performed, and the procedure terminates. On the other hand, if at step 704 it is determined that the variable i is not greater or equal to the total number of testing units TUtot, then at step 705 the variable i is incremented by a factor of 1, and the step of providing power to the subsequent testing unit in the manufacturing process batch at step 703 is performed.

Alternatively, referring back to FIG. 7, at step 703, if the variable i is determined to be equal to or greater than the total number of testing units TUtot, then it may be determined that one charging cycle of the testing units TUi have been completed. Thus, in the case where the testing process has not been completed, the routine may be repeated for additional charging cycles to provide power to the testing units TUi to complete the testing procedures of the respective sensors during the manufacturing and testing cycle.

In this manner, in one embodiment of the present invention, the testing of each of the sensors in the tester isolation system 100 may be performed so that the testing procedure for each sensor is performed to completion. For example, for a manufacturing batch with 10 testing channels, each channel provided with a respective testing unit TU coupled a corresponding sensor, and with 500 ms cycle per channel, each channel will be charged at five second intervals. This process continues until the testing procedure is completed. Indeed, each testing channel continues to perform the testing routines between the five second recharge intervals since it has stored energy from the previous charging process.

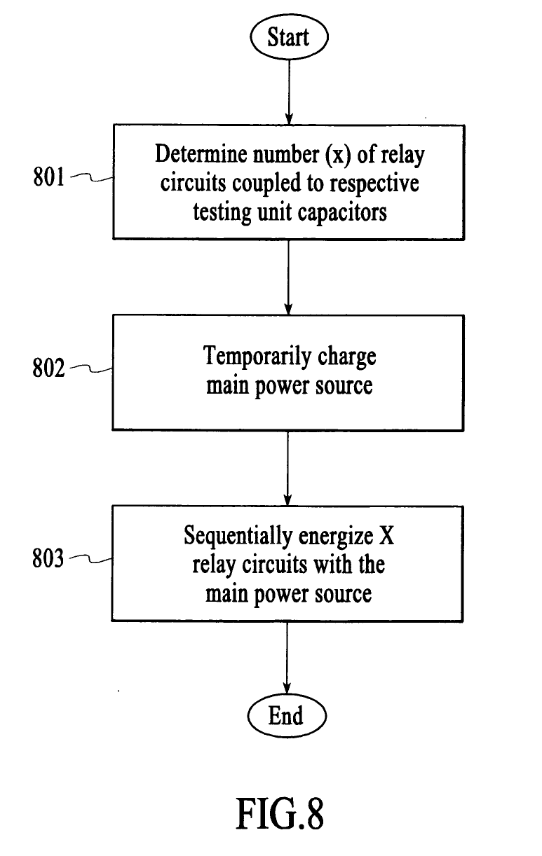

FIG. 8 is a flow chart illustrating the sensor tester isolation process in accordance with another embodiment of the present invention. Referring to FIG. 8, at step 801, sensor tester isolation system in one embodiment of the present invention is configured to determine the number (X) of the relay circuits coupled to a respective one of the testing unit capacitors. Thereafter at step 802, a main power source such as a capacitor is charged using, for example, a power source (Vsource) and disconnected after charging. Thereafter, at step 803, the main power source is configured to sequentially energize the X number of relay circuits so as to sequentially power the respective testing units to perform the respective testing procedures on the respective analyte sensors coupled thereto.

Within the scope of the present invention, the steps 801 to 803 may be repeated to perform the testing procedures on multiple batches of analyte sensors to provide scalability and efficiency in analyte sensor manufacturing and testing processes.

In this manner, the sensor tester isolation system in one embodiment of the present invention is configured to sequentially power the tester circuits coupled to each of the analyte sensors in the testing batch that are immersed in the electrically conductive test fluid such that electrical isolation may be achieved as between the sensors in the testing batch such that efficient, multiple sensor testing procedures may be performed, while minimizing potential testing errors or interferences by potential currents between the sensors in the test fluid.

Indeed, within the scope of the present invention, by providing electrical isolation between adjacent test channels for the sensors under testing procedure, it is possible to perform test procedures at the same time on multiple biosensors for use in, for example, analyte monitoring systems, so that many sensors can be tested at the same time in production environment. In this manner, manufacturing efficiency may be achieved by simultaneously performing test procedures on multiple sensors, rather than one at a time, or alternatively, risk potential errors in sensor testing output if multiple sensor testing is performed simultaneously due to the potentially presence of current between the sensors which may interfere with the accuracy of the testing procedures.

In this manner, a system for providing electrical isolation in one embodiment of the present invention includes a power source, a controller unit coupled to the power source, a plurality of sensor testing units, and plurality of switches each operatively coupled to a respective one of the plurality of sensor testing units, the plurality of switches further operatively coupled to the controller unit, where the controller unit is configured to selectively couple a respective one or more of the plurality of switches to the power source.

The power source in one embodiment may include a capacitor, for example, of 1.0 Farad.

In a further embodiment, each of the plurality of switches may include a relay circuit.

Moreover, each of the plurality of sensor testing units may be operatively coupled to a respective one of a plurality of sensors, where the controller unit may be configured to provide electrical isolation between the plurality of sensors. The sensors may include analyte sensors, and in particular, may include glucose sensors.

Also, each of the plurality of sensors may be configured for fluid contact with an analyte, where the analyte includes one of interstitial fluid, blood, or oxygen.

A system for providing electrical isolation in a further embodiment includes a plurality of sensor testing units, each sensor testing unit including a power source, a plurality of switches each operatively coupled to a respective one of the plurality of sensor testing units, and a controller unit operatively coupled to the plurality of switches, the controller unit further configured to sequentially trigger the plurality of switches such that the plurality of sensor testing units are each configured to receive power from the respective power source.

In one embodiment, each of the sensor testing units may be operatively coupled to a respective one of a plurality of sensors, where the plurality of sensors is configured to be electrically isolated.

The plurality of switches may include a plurality of relay switches.

In addition, the power source for each of the plurality of sensor testing units may include a capacitor.

A method in yet another embodiment of the present invention includes charging a power source, selectively coupling the power source to a plurality of sensor testing units, maintaining electrical isolation between the plurality of sensor testing units.

In one embodiment, the step of charging the power source may include the steps of coupling a capacitance to a power supply, and disconnecting the capacitance from the power supply.

Further, the step of selectively coupling may include the step of selectively providing power to the plurality of sensor testing units.

Moreover, the step of maintaining electrical isolation may include the step of electrically separating each of the plurality of sensor testing units.

Additionally, the method may further include the step of coupling a respective sensor to each of the plurality of sensor testing units.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

What is claimed is:

1. A system for providing electrical isolation, comprising:
a power source;
a controller unit coupled to the power source;
a plurality of sensor testing units; and
a plurality of switches each operatively coupled to a respective one of the plurality of sensor testing units, the plurality of switches further operatively coupled to the controller unit;
wherein the controller unit is configured to selectively couple a respective one or more of the plurality of switches to the power source.

2. The system of claim 1 wherein the power source includes a capacitor.

3. The system of claim 2 wherein the capacitor is about 1.0 Farad.

4. The system of claim 1 wherein each of the plurality of switches is a relay circuit.

5. The system of claim 1 wherein each of the plurality of sensor testing units is operatively coupled to a respective one of a plurality of sensors.

6. The system of claim 6 wherein at least one of the plurality of sensors is an analyte sensor.

7. The system of claim 5 wherein at least one of the plurality of sensors is a glucose sensor.

8. The system of claim 5 wherein the controller unit is configured to provide electrical isolation between the plurality of sensors.

9. The system of claim 5 wherein each of the plurality of sensors is configured for fluid contact with an analyte.

10. The system of claim 9 wherein the analyte includes one of interstitial fluid, blood, or oxygen.

11. A system for providing electrical isolation, comprising:
a plurality of sensor testing units, each sensor testing unit including a power source;
a plurality of switches each operatively coupled to a respective one of the plurality of sensor testing units; and
a controller unit operatively coupled to the plurality of switches, the controller unit further configured to sequentially trigger the plurality of switches such that the plurality of sensor testing units are each configured to receive power from the respective power source.

12. The system of claim 11 wherein each of the sensor testing units are operatively coupled to a respective one of a plurality of sensors.

13. The system of claim 12 wherein at least one of the plurality of sensors is an analyte sensor

14. The system of claim 12 wherein at least one of the plurality of sensors is a glucose sensor.

15. The system of claim 12 wherein the plurality of sensors are configured to be electrically isolated.

16. The system of claim 12 wherein each of the plurality of sensors is configured for fluid contact with an analyte.

17. The system of claim 16 wherein the analyte includes one of interstitial fluid, blood, or oxygen.

18. The system of claim 11 wherein the plurality of switches includes a plurality of relay switches.

19. The system of claim 11 wherein the power source for each of the plurality of sensor testing units is a capacitor.

20. A method, comprising:
charging a power source;
selectively coupling the power source to a plurality of sensor testing units; and
maintaining electrical isolation between the plurality of sensor testing units.

21. The method of claim 20 wherein the step of charging the power source includes the steps of:
coupling a capacitance to a power supply; and
disconnecting the capacitance from the power supply.

22. The method of claim 20 wherein the step of selectively coupling includes the step of selectively providing power to the plurality of sensor testing units.

23. The method of claim 20 wherein the step of maintaining electrical isolation includes the step of electrically separating each of the plurality of sensor testing units.

24. The method of claim 20 further including the step of coupling a respective sensor to each of the plurality of sensor testing units.

25. The method of claim 20 further including the step of coupling a sensor to a respective one of the plurality of sensor testing units.

26. The method of claim 25 further including the step of maintaining electrical isolation between each sensor coupled to a respective one of the plurality of sensor testing units.

27. The method of claim 25 wherein the sensor includes an analyte sensor.

28. The method of claim 25 wherein the sensor includes a glucose sensor.
